(19) United States
(12) Patent Application Publication
(10) Pub. No.: US 2018/0079705 A1
(43) Pub. Date: Mar. 22, 2018

(54) METHOD FOR PURIFICATION OF AN AROMATIC DIACID OR THE CORRESPONDING ANHYDRIDE

(71) Applicant: SABIC Global Technologies B.V., Bergen op Zoom (NL)

(72) Inventors: Vinodkumar Vasudevan, Riyadh (SA); Guillermo Leal, Riyadh (SA); Nedumbamana Sankaran, Riyadh (SA); Syed Azhar Hashmi, Riyadh (SA)

(21) Appl. No.: 15/562,240

(22) PCT Filed: Mar. 18, 2016
(86) PCT No.: PCT/IB2016/051553

§ 371 Date: Sep. 27, 2017

Related U.S. Application Data
(60) Provisional application No. 62/140,560, filed on Mar. 31, 2015.

(51) Int. Cl. C07C 51/43; C07C 249/08; C07C 63/331

(52) U.S. Cl. CPC C07C 51/43; C07B 2200/13; C07C 63/331; C07C 249/08

(57) ABSTRACT

A method of purifying a stream having an aromatic carboxy-aldehyde and an aromatic acid or the corresponding anhydride is described. The method includes reacting a hydroxylamine-containing compound with the aromatic carboxy-aldehyde in the stream to form a reaction mixture including the corresponding nitrone; adding an aqueous solvent to the reaction mixture, in an amount and under conditions effective to solubilize the nitrone but not the organic acid or the corresponding anhydride; and separating the solubilized nitrone from the non-solubilized organic acid. The nitrone is a water soluble nitrone.

### BACKGROUND

Aromatic acids, for example aromatic carboxylic acids, are important intermediates for the preparation of linear polymers useful for films, fibers, and the like. Of particular importance are aromatic dicarboxylic acids such as phthalic acid, terephthalic acid, and isophthalic acid for use in the manufacture of synthetic polymers. These intermediates must be exceptionally free of impurities which are colored, which may become colored, or which can act as chain terminators in the polymerization step thereby causing low quality polymers to be obtained. These impurities generally arise during the preparation of the dicarboxylic acid from the starting hydrocarbon (e.g., xylene). Due to the physical and chemical properties of the impurities, known purification and separation processes such as re-crystallization, distillation, and sublimation are generally ineffective for purifying the aromatic acids.

Alternative methods for purifying the aromatic acids include separating and purifying the phthalic acids by way of the corresponding dimethyl esters. The corresponding dimethyl esters can be obtained with high purity following several recrystallization and/or distillation steps. However, if the free acids are required, the esters must be saponified after the purification. Therefore, while the aforementioned process can provide pure phthalic acids, it is not economical.

There has been an active interest in overcoming the above-described technical limitations for purifying aromatic carboxylic acids. Accordingly, there remains a need for an improved purification and separation process for the aromatic carboxylic acids.

### BRIEF DESCRIPTION

A method of purifying a stream comprising an aromatic carboxy-aldehyde and an aromatic acid or the corresponding anhydride comprises: reacting a hydroxylamine-containing compound with the aromatic carboxy-aldehyde in the stream to form a reaction mixture comprising the corresponding nitrone, wherein the nitrone is water soluble; adding an aqueous solvent to the reaction mixture, in an amount and under conditions effective to solubilize the nitrone but not the aromatic acid or the corresponding anhydride; and separating the solubilized nitrone from the non-solubilized aromatic acid.

A method for purifying a phenyl dicarboxylic acid or the corresponding anhydride comprises: oxidizing a xylene to provide a stream comprising the phenyl dicarboxylic acid and the corresponding carboxybenzaldehyde and toluic acid contaminants; reacting a hydroxylamine-containing compound with the carboxybenzaldehyde in the stream to form a reaction mixture comprising the corresponding nitrone, wherein the nitrone is water soluble; and crystallizing the phenyl dicarboxylic acid or the corresponding anhydride from water to provide the purified phenyl dicarboxylic acid or the corresponding anhydride.

These and other features and characteristics are more particularly described below.

### BRIEF DESCRIPTION OF THE DRAWINGS

The following is a brief description of the drawings wherein like elements are numbered alike and which are presented for the purposes of illustrating the exemplary embodiments disclosed herein and not for the purposes of limiting the same.

FIG. 1 is a schematic drawing illustrating the method for manufacturing phenyl dicarboxylic acid.

FIG. 2 is a schematic drawing of a system for manufacturing phenyl dicarboxylic acid.

FIG. 3 is an illustration of a chemical scheme illustrating the formation of a water-soluble nitrone from a carboxy-aldehyde impurity and a hydroxy-amine-containing compound.

### DETAILED DESCRIPTION

Described herein is a method for removing an aromatic carboxy-aldehyde from an aromatic acid and a method for the manufacture of a phenyl dicarboxylic acid. An aromatic acid or the corresponding anhydride prepared according to the method represents another aspect of the disclosure. A phenyl dicarboxylic acid prepared according to the method is also described. It was unexpectedly discovered that a carboxy-aldehyde impurity could be reacted with a hydroxylamine-containing compound to form the corresponding nitrone. The corresponding nitrone can be solubilized in water, and separated from the aromatic acid by filtering, decanting, centrifuging, or the like. Advantageously, no additional purification steps (e.g., recrystallization, hydrogenation, esterification, and the like) are necessary. The method disclosed herein can provide the aromatic acid at high purity, for example, 90 to 95%. The aromatic acid can also have reduced yellowness index compared to the aromatic acid comprising the aromatic carboxy-aldehyde impurity.

A method of removing an aromatic carboxy-aldehyde from an aromatic acid or the corresponding anhydride can include reacting a hydroxylamine-containing compound with the aromatic carboxy-aldehyde to form a reaction mixture comprising the corresponding nitrone. The initial mixture comprising the aromatic carboxy-aldehyde and the aromatic acid or the corresponding anhydride can be the product of a xylene oxidation, for example, a xylene oxidation conducted on a scale of at least 1,000 kilograms per hour. The xylene oxidation can be, for example, a liquid phase oxidation of xylene with air, oxygen, or an oxygen-containing gas. The liquid phase oxidation process can include a solvent, for example acetic acid. The oxidation can take place at a temperature of, for example, 150 to 225° C., and a pressure of, for example, about 2.0 MegaPascals (MPa). In some embodiments, the xylene oxidation process can include use of a catalyst, for example a catalyst comprising cobalt ions, manganese ions, bromide ions, or a combination comprising at least one of the foregoing.

The aromatic carboxy-aldehyde can be removed from the aromatic acid or corresponding anhydride by reacting a hydroxylamine-containing compound with the aromatic carboxy-aldehyde. The reacting of the hydroxylamine-containing compound with the aromatic carboxy-aldehyde can be in the presence of the aromatic acid or corresponding anhydride. The hydroxylamine-containing compound has the structure

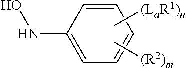

wherein n is 1 to 5 and m is 0 to 4, provided that n+m is not greater than 5. For example, n+m can be 1, 2, 3, 4, or 5. In some embodiments, a is 0 or 1. L is a divalent C₁-C₃ alkylene or phenylene group.

Each occurrence of R¹ can independently be a hydroxyl, sulfhydryl, sulfonyl, sulfonic acid or a salt thereof, tosylate, mesylate, C₂-C₅ alkoxycarbonyl, C₂-C₅ alkylcarbonyloxy, C₂-C₅ carboxamide, alkoxycarbonyl, C₂-C₅ alkylcarbonyloxy, phosphonic acid or a salt thereof, phosphoric acid or a salt thereof, carboxylic acid or a salt thereof, C₁-C₄ alkylthio, ether of the formula —OR^{a}, amino of the formula —NR^{b}R^{c}, aminocarboxy of the formula —C(O)NR^{b}R^{c}, quaternary phosphonium salt of the formula —P⁺R^{b}R^{c}R^{d}X⁻, or quaternary ammonium salt of the formula —N⁺R^{b}R^{c}R^{d}X. Each occurrence of R^{a} can independently be C₁-C₄ alkyl or —(CᵣH₂ᵣO)ₛCᵣH₂ᵣ₊₁ wherein each occurrence of r is independently 1 to 3 and each occurrence of s is independently 1 to 12. Each occurrence of R^{b}, R^{c}, and R^{d} can independently be hydrogen, C₁-C₄ alkyl, hydroxy-substituted C₁-C₄ alkylene, C₁-C₄ alkoxy-substituted C₁-C₄ alkylene, amino(C₁-C₄ alkylene), or N,N-(di-C₁-C₃ alkyl)amino-C₁-C₄ alkylene. Each occurrence of X can be an organic or inorganic ion, for example a hydroxide, halide, carboxylate, sulfonate, sulfate, formate, carbonate, or bicarbonate. Where X⁻ is a polyvalent anion such as carbonate or sulfate it is understood that the positive and negative charges in the quaternary ammonium and phosphonium structures are properly balanced. Examples of organic quaternary ammonium compounds include tetramethyl ammonium hydroxide, tetrabutyl ammonium hydroxide, tetramethyl ammonium acetate, tetramethyl ammonium formate, tetrabutyl ammonium acetate, and combinations comprising at least one of the foregoing. Examples of organic quaternary phosphonium compounds include tetramethyl phosphonium hydroxide, tetramethyl phosphonium acetate, tetramethyl phosphonium formate, tetrabutyl phosphonium hydroxide, tetrabutyl phosphonium acetate (TBPA), tetraphenyl phosphonium acetate, tetraphenyl phosphonium phenoxide, and combinations comprising at least one of the foregoing.

Each occurrence of R² can independently be halogen, cyano, thiocyanato, nitro, C₁-C₃ alkyl, C₂-C₃ alkenyl, C₁-C₃ alkoxy, C₁-C₃ alkylthio, C₃-C₅ cycloalkyl, C₂-C₅ acyl, C₃-C₈ heteroaryl, or carbamoyl.

In some embodiments, each occurrence of R¹ is independently a hydroxyl, sulfonic acid or a salt thereof, phosphonic acid or a salt thereof, phosphoric acid or a salt thereof, carboxylic acid or a salt thereof, amino group of the formula —NR^{b}R^{c}, quaternary phosphonium salt of the formula —P⁺R^{b}R^{c}R^{d}X—, or quaternary ammonium salt of the formula —N⁺R^{b}R^{c}R^{d}X⁻, wherein each occurrence of R^{a}, R^{b}, and R^{c} is independently hydrogen, C₁-C₄ alkyl, hydroxy-substituted C₁-C₄ alkylene, C₁-C₄ alkoxy-substituted C₁-C₄ alkylene, amino(C₁-C₄ alkylene), or N,N-(di-C₁-C₃ alkyl)amino-C₁-C₄ alkylene.

In some embodiments, each occurrence of R¹ is independently a hydroxyl, sulfonic acid or a salt thereof, phosphonic acid or a salt thereof, phosphoric acid or a salt thereof, carboxylic acid or a salt thereof, quaternary phosphonium salt of the formula —P⁺R^{b}R^{c}R^{d}X⁻, or quaternary ammonium salt of the formula —N⁺R^{b}R^{c}R^{d}X⁻, wherein each occurrence of R^{a}, R^{b}, and R^{c} is independently hydrogen or C₁-C₄ alkyl.

In some embodiments, the hydroxylamine-containing compound can be present in an amount of 0.001 to 10 weight percent, based on the weight of the aromatic carboxy-aldehyde, for example, 0.05 to 5 weight percent, for example, 0.1 to 2.5 weight percent.

The aromatic carboxy-aldehyde can have the structure

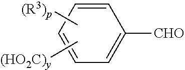

wherein p is 0 to 4 and y is 1 to 5, provided that p+y is not greater than 5. For example, p+y can be 1, 2, 3, 4, or 5. Each occurrence of R³ is independently halogen, cyano, thiocyanato, nitro, C₁-C₅ alkyl, C₁-C₅ alkoxy, C₁-C₅ alkylthio, C₃-C₅ cycloalkyl, C₂-C₅ acyl, C₆-C₁₂ aryl, C₆-C₁₂ aryloxy, C₃-C₅ heteroaryl, or carbamoyl. In some embodiments, p is 0, and y is 1 to 5, preferably y is 1. For example, the aromatic carboxy-aldehyde can comprise 2-carboxybenzaldehyde, 3-carboxybenzaldehyde, 4-carboxybenzaldehyde, or a combination comprising at least one of the foregoing.

The aromatic acid can comprise at least 2 carboxylic acid groups. For example, the aromatic acid can comprise 2, 3, 4, or 5 carboxylic acid groups. In some embodiments, the aromatic acid is a dicarboxylic acid, for example phthalic acid, terephthalic acid, isophthalic acid, or a combination comprising at least one of the foregoing. In an embodiment, the corresponding anhydride is phthalic anhydride. In some embodiments, the aromatic acid is a tricarboxylic acid, for example benzene-1,3,5-tricarboxylic acid.

The hydroxylamine-containing compound can react with the aromatic carboxy-aldehyde to form a reaction mixture comprising the corresponding nitrone. In some embodiments, the nitrone is water soluble. For example, the nitrone can have a water solubility at 25° C. of greater than or equal to 1 gram per 100 milliliters (g/100 mL), preferably greater than or equal to 2 g/100 mL, preferably greater than or equal to 5 g/100 mL. The nitrone has the structure

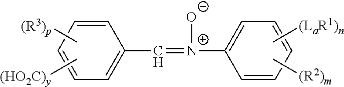

wherein p is 0 to 4, y is 1 to 5, provided that p+y is not greater than 5. For example p+y can equal 1, 2, 3, 4, or 5. In some embodiments, n is 1 to 5 and m is 0 to 4, provided that n+m is not greater than 5. For example n+m can equal 1, 2, 3, 4, or 5. In some embodiments, a is 0 or 1 and L is a C₁-C₃ alkylene or phenylene. In some embodiments, each occurrence of R¹ is independently a hydroxyl, sulfhydryl, sulfonyl, C₂-C₅ alkoxycarbonyl, C₂-C₅ alkylcarbonyloxy, sulfonic acid, carboxamide or a salt thereof, tosylate, mesylate, phosphonic acid or a salt thereof, phosphoric acid or a salt thereof, carboxylic acid or a salt thereof, C₂-C₄ alkylamido, C₁-C₄ alkylthio, ether of the formula —OR^{a}, amino group of the formula —NR^{b}R^{c}, quaternary phosphonium salt of the formula —P⁺R^{b}R^{e}R^{d}X⁻, or quaternary ammonium salt of the formula —N⁺R^{b}R^{e}R^{d}X⁻, wherein each occurrence of R^{a} is independently C₁-C₄ alkyl or —(CᵣH₂ᵣO)ₛCᵣH₂ᵣ₊₁ wherein each occurrence of r is independently 1 to 3 and each occurrence of s is independently 1 to 12, wherein each occurrence of R^{b}, R^{c}, and R^{d} is independently hydrogen, C₁-C₄ alkyl, C₂-C₄ alkenyl, hydroxy-substituted C₁-C₄ alkylene, C₁-C₄ alkoxy-substituted C₁-C₄ alkylene, amino(C₁-C₄ alkylene), N,N-(di-C₁-C₃ alkyl)amino-C₁-C₄ alkylene, and each occurrence of X is an organic or inorganic ion. In some embodiments, each occurrence of R² is independently halogen, cyano, thiocyanato, nitro, C₁-C₃ alkyl, C₂-C₃ alkenyl, C₁-C₃ alkoxy, C₁-C₃ alkylthio, C₃-C₅ cycloalkyl, C₂-C₅ acyl, C₃-C₈ heteroaryl, or carbamoyl. In some embodiments, each occurrence of R³ is independently halogen, cyano, thiocyanato, nitro, C₁-C₅ alkyl, C₁-C₅ alkenyl, C₁-C₅ alkoxy, C₁-C₅ alkylthio, C₃-C₅ cycloalkyl, C₂-C₅ acyl, C₆-C₁₂ aryl, C₆-C₁₂ aryloxy, C₃-C₈ heteroaryl, or carbamoyl.

The method of purifying a stream comprising an aromatic carboxy-aldehyde and an aromatic acid or the corresponding anhydride can further include adding an aqueous solvent to the reaction mixture. The aqueous solvent can be added in an amount and under conditions effective to solubilize the nitrone but not the aromatic acid or corresponding anhydride. For example, the aqueous solvent can be added to the reaction mixture in a solvent:reaction mixture volumetric ratio of 1:100 to 100:1, for example 1:50 to 50:1, for example 1:20 to 20:1, for example 1:10 to 10:1, for example 1:5 to 5:1, for example 1:2 to 2:1, for example 1:1 to solubilize the nitrone but not the aromatic acid or the corresponding anhydride. For example, the aqueous solvent can be added to the reaction mixture at a temperature of 0 to 150° C., for example 10 to 100° C., for example 20 to 75° C., for example 20 to 50° C. The aqueous solvent can comprise water. In some embodiments, the aqueous solvent is devoid of organic solvent, for example comprises less than 1 wt. %, for example 0.5 wt. % of an organic solvent. In some embodiments, the aqueous solvent can further comprise salts (i.e., ions). For example, the aqueous solvent can comprise alkali metal salts (e.g., sodium chloride, potassium chloride, and the like). For the example the aqueous solvent can comprise a polyelectrolyte (e.g., polyacrylic acid). The aqueous solvent can further comprise water-miscible solvents, for example, C₁-C₆ alcohols. In some embodiments, the aqueous solvent can be an aqueous mineral acid such as hydrochloric acid, nitric acid, phosphoric acid, sulfuric acid, boric acid, hydrofluoric acid, hydrobromic acid, perchloric acid, or a combination comprising at least one of the foregoing. The aqueous solvent can also be an aqueous organic acid that includes a carboxylic acid, sulfonic acid, or a combination comprising at least one of the foregoing. Exemplary carboxylic acids include formic acid, acetic acid, chloroacetic acid, dichloroacetic acid, trichloroacetic acid, trifluoroacetic acid, propionic acid, butyric acid, oxalic acid, benzoic acid, phthalic acid (including ortho-, meta- and para-isomers), and the like. Exemplary sulfonic acids include a C₁C-₂₀ alkyl sulfonic acid, wherein the alkyl group can be branched or unbranched and can be substituted or unsubstituted, or a C₃-C₂₀ aryl sulfonic acid wherein the aryl group can be monocyclic or polycyclic, and optionally comprises 1 to 3 heteroatoms (e.g., N, S, or P). Alkyl sulfonic acids can include, for example, methane sulfonic acid. Aryl sulfonic acids include, for example, benzene sulfonic acid or toluene sulfonic acid. In some embodiments, the aryl group can be C₁-C₂₀ alkyl-substituted, i.e., is an alkylarylene group, or is attached to the sulfonic acid moiety via a C₁-C₂₀ alkylene group (i.e., an arylalkylene group), wherein the alkyl or alkylene can be substituted or unsubstituted. In an embodiment, the aqueous solvent is water.

The method can further include separating the solubilized nitrone from the non-solubilized aromatic acid. In some embodiments, adding the solvent and separating can comprise adding an aqueous solvent comprising water, and crystallizing the aromatic acid or the corresponding anhydride from the aqueous solvent. In some embodiments, separating the solubilized nitrone from the non-solubilized aromatic acid can be by, for example, filtering, decanting, centrifuging, and the like. In some embodiments, following separation, the isolated aromatic acid or the corresponding anhydride can be crystallized to remove the corresponding toluic acid.

The separated aromatic acid or the corresponding anhydride can have a purity of 90 to 99.9%, for example 90 to 99%, for example, 90 to 95%. For example, in some embodiments, the separated aromatic acid or the corresponding anhydride can have less than 2 weight percent (wt. %) of the aromatic carboxy-aldehyde, for example less than 1 wt. % of the aromatic carboxy-aldehyde, for example less than 0.5 wt. % of the carboxy aldehyde.

The crystallized aromatic acid or the corresponding anhydride can have less than 2 wt. % of the corresponding toluic acid, for example, less than 1 wt. % of the corresponding toluic acid, for example less than 0.5 wt. % of the corresponding toluic acid.

In some embodiments, the separated aromatic acid or the corresponding anhydride can have a yellowness index of less than or equal to 10, preferably less than or equal to 5, more preferably less than or equal to 2, as determined according to ASTM D1209.

In some embodiments, after the reacting to form the nitrone, or after isolating the purified aromatic acid or corresponding anhydride, the aromatic acid or corresponding anhydride can have a delta Y value at least 5% lower, for example at least 10% lower, for example, at least 20% lower than the aromatic acid or corresponding anhydride before the reacting to form the nitrone, as determined according to ASTM D1209. In some embodiments, the color properties (e.g., yellowness index) of the purified aromatic acid or corresponding anhydride can be evaluated using a tristimulus colorimeter.

Also described herein is a method for the manufacture of a phenyl dicarboxylic acid or the corresponding anhydride. The method can include oxidizing a xylene to provide a stream comprising the phenyl dicarboxylic acid and the corresponding carboxybenzaldehyde and toluic acid contaminants; reacting a hydroxylamine-containing compound with the carboxybenzaldehyde in the stream to form a reaction mixture comprising the corresponding nitrone, wherein the nitrone is water soluble; and crystallizing the phenyl dicarboxylic acid or the corresponding anhydride from water to provide the purified phenyl dicarboxylic acid or the corresponding anhydride.

The xylene can be ortho-xylene, meta-xylene, para-xylene, or a combination comprising at least one of the foregoing xylenes. In some embodiments, the xylene is a combination comprising at least two of the foregoing xylenes.

The hydroxylamine-containing compound can be as previously described herein. The phenyl dicarboxylic acid can include 2 carboxylic acid groups. For example, the phenyl dicarboxylic acid can be phthalic acid, terephthalic acid, isophthalic acid, or a combination comprising at least one of the foregoing.

The phenyl dicarboxylic acid or the corresponding anhydride can comprise less than or equal to 0.5 wt. % of the carboxybenzaldehyde, for example less than or equal to 0.25 wt. % of the carboxybenzaldehyde, for example less than or equal to 0.05 wt. % of carboxybenzaldehyde. The phenyl dicarboxylic acid or the corresponding anhydride can comprise less than or equal to 0.2 wt. % of the toluic acid, for example less than or equal to 0.1 wt. % of the toluic acid, for example less than or equal to 0.05 wt. % of the toluic acid.

In some embodiments, the method advantageously excludes a purification step including a hydrogenation reaction. For example, the method for manufacturing a phenyl dicarboxylic acid can exclude the catalytic hydrogenation of the aromatic carboxy-aldehyde, for example using a palladium hydrogenation catalyst.

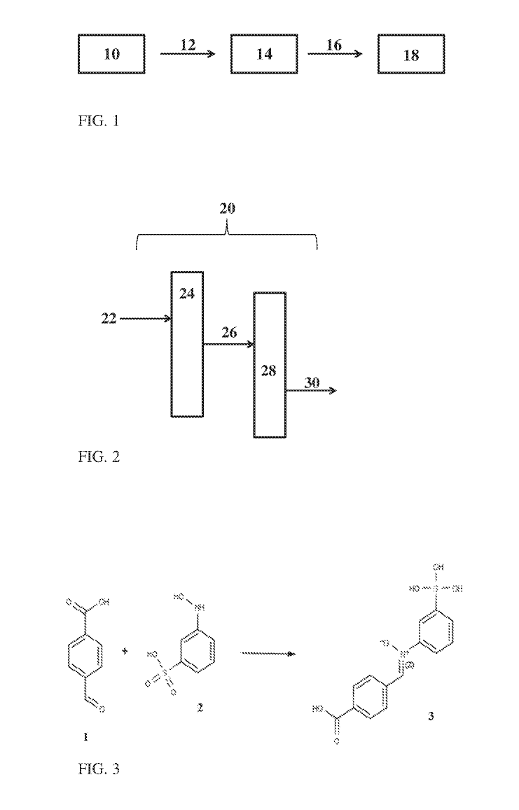

The method for manufacturing the phenyl dicarboxylic acid as described above is further illustrated in FIG. 1. Xylene 10 is oxidized 12 to provide a crude phenyl dicarboxylic acid product 14 comprising the phenyl dicarboxylic acid and the corresponding carboxybenzaldehyde and toluic acid contaminants. The crude product 14 is purified 16, for example by reacting a hydroxylamine-containing compound with the carboxybenzaldehyde to form the corresponding nitrone. A purified phenyl dicarboxylic acid product 18 is obtained by crystallizing the phenyl dicarboxylic acid or the corresponding anhydride from water to provide the purified phenyl dicarboxylic acid or the corresponding anhydride 18.

As illustrated in FIG. 2, a system 20 for the manufacture of a phenyl dicarboxylic acid according to method disclosed herein represents another aspect of the present disclosure. The system 20 can comprise a feed stream 22 comprising xylene, acetic acid, and a catalyst entering into a first reactor 24 for oxidizing a xylene to provide a stream 26 comprising the phenyl dicarboxylic acid and the corresponding carboxybenzaldehyde and toluic acid contaminants, and a second reactor 28 for reacting a hydroxylamine-containing compound with the carboxybenzaldehyde in the stream 26 to form a reaction mixture 30 comprising the corresponding nitrone. The first reactor can generally be any reactor for carrying out a liquid phase oxidation of xylene. For example, the first reactor can be a continuous or semi-continuous stirred tank reactor, a batch reactor, a tower reactor, a tubular reactor, or a multitubular reactor. Any of the aforementioned reactors can be employed in series or in parallel. In some embodiments, reacting a hydroxylamine-containing compound with the carboxybenzaldehyde can be carried out in a second reactor different from the first reactor. In some embodiments, reacting a hydroxylamine-containing compound with the carboxybenzaldehyde can be carried out in the same reactor as used for oxidizing the xylene (i.e., the first and second reactors can be the same or different reactors).

The following example is merely illustrative of the method disclosed herein and are not intended to limit the scope hereof.

### Example

An aromatic carboxy-aldehyde impurity was removed from a phenyl carboxylic acid according to the above described method, and as shown in the chemical scheme of FIG. 3.

Following the oxidation of xylene, 3-(hydroxyamino)benzene-1-sulfonic acid (shown as compound 2 in FIG. 3) was added to the reactor in a molar amount equivalent to the molar amount of carboxy-aldehyde impurity (shown as compound 1 in FIG. 3). After addition of compound 2, the reaction mixture was stirred for 1 hour at a temperature of about 20 to about 50° C. After 1 hour, the reaction mixture was filtered to remove the nitrone (shown as compound 3 in FIG. 3). Additional water was added to completely remove the water-soluble nitrone. Following the separation, purified phenyl carboxylic acid was obtained.

The method disclosed herein includes at least the following embodiments:

### Embodiment 1

A method of purifying a stream comprising an aromatic carboxy-aldehyde and an aromatic acid or the corresponding anhydride, comprising: reacting a hydroxylamine-containing compound with the aromatic carboxy-aldehyde in the stream to form a reaction mixture comprising the corresponding nitrone, wherein the nitrone is water soluble; adding an aqueous solvent to the reaction mixture, in an amount and under conditions effective to solubilize the nitrone but not the aromatic acid or the corresponding anhydride; and separating the solubilized nitrone from the non-solubilized aromatic acid.

### Embodiment 2

The method of Embodiment 1, wherein the adding the solvent and the separating comprises crystallizing the aromatic acid or the corresponding anhydride from the aqueous solvent.

### Embodiment 3

The method of Embodiment 2, wherein the solvent is water.

### Embodiment 4

The method of Embodiment 3, wherein the crystallizing further removes the corresponding toluic acid from the aromatic acid or the corresponding anhydride.

### Embodiment 5

The method of any of Embodiments 1 to 4, wherein the separating is by filtering, decanting, or centrifuging the reaction mixture to separate the aromatic acid or the corresponding anhydride from the solvent comprising the solubilized nitrone.

### Embodiment 6

The method of Embodiment 5, further comprising crystallizing the separated aromatic acid or the corresponding anhydride to remove the corresponding toluic acid.

### Embodiment 7

The method of any of Embodiments 1 to 6, wherein the separated aromatic acid or corresponding anhydride has a purity of 90-99%.

### Embodiment 8

The method of any of Embodiments 2 to 3 or 6, wherein the crystallized aromatic acid or corresponding anhydride has less than 2 wt. % of the corresponding toluic acid, preferably less than 1 wt. % of the corresponding toluic acid, more preferably less than 0.5 wt. % of the corresponding toluic acid.

### Embodiment 9

The method of any of Embodiments 2 to 3 or 6 to 8, wherein after the reacting to form the nitrone or the separating, the aromatic acid or corresponding anhydride has a yellowness index of less than or equal to 10, preferably less than or equal to 5, more preferably less than or equal to 2 as determined according to ASTM D1209.

### Embodiment 10

The method of any of Embodiments 2 to 3 or 6 to 9, wherein after the reacting to form the nitrone or the separating, the aromatic acid or corresponding anhydride has a delta-Y value of at least 5% lower, at least 10% lower, or at least 20% lower than the aromatic acid or corresponding anhydride before the reacting, as determined according to ASTM D1209.

### Embodiment 11

The method of any of Embodiments 1 to 10, wherein the stream comprising an aromatic carboxy-aldehyde and an aromatic acid or the corresponding anhydride are the product of a xylene oxidation.

### Embodiment 12

The method of Embodiment 11, wherein the xylene oxidation is conducted on a scale of at least 1,000 kilograms per hour.

### Embodiment 13

The method of any of Embodiments 1 to 12, wherein the hydroxylamine-containing compound has the structure

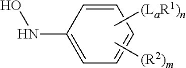

wherein n is 1 to 5 and m is 0 to 4, provided that n+m=1, 2, 3, 4, or 5, a is 0 or 1, L is a C₁-C₃ alkylene or phenylene, each occurrence of R¹ is independently a hydroxyl, sulfhydryl, sulfonyl, sulfonic acid or a salt thereof, tosylate, mesylate, C₂-C₅ alkoxycarbonyl, C₂-C₅ alkylcarbonyloxy, C₂-C₅ carboxamide, alkoxycarbonyl, C₂-C₅ alkylcarbonyloxy, phosphonic acid or a salt thereof, phosphoric acid or a salt thereof, carboxylic acid or a salt thereof, C₁-C₄ alkylthio, ether of the formula —OR^{a}, amino of the formula —NR^{b}R^{c}, aminocarboxy of the formula —C(O)NR^{b}R^{c}, quaternary phosphonium salt of the formula —P⁺R^{b}R^{c}R^{d}X⁻, or quaternary ammonium salt of the formula —N⁺R^{b}R^{c}R^{d}X⁻; wherein each occurrence of R^{a} is independently C₁-C₄ alkyl or —(CᵣH₂ᵣO)ₛCᵣH₂ᵣ₊₁ wherein each occurrence of r is independently 1 to 3 and each occurrence of s is independently 1 to 12, wherein each occurrence of R^{b}, R^{c}, and R^{d} is independently hydrogen, C₁-C₄ alkyl, hydroxy-substituted C₁-C₄ alkylene, C₁-C₄ alkoxy-substituted C₁-C₄ alkylene, amino(C₁-C₄ alkylene), N,N-(di-C₁-C₃ alkyl)amino-C₁-C₄ alkylene, and each occurrence of X is an organic or inorganic ion, and each occurrence of R² is independently halogen, cyano, thiocyanato, nitro, C₁-C₃ alkyl, C₂-C₃ alkenyl, C₁-C₃ alkoxy, C₁-C₃ alkylthio, C₃-C₅ cycloalkyl, C₂-C₅ acyl, C₃-C₈ heteroaryl, or carbamoyl.

### Embodiment 14

The method of Embodiment 13, wherein each occurrence of R¹ is independently a hydroxyl, sulfonic acid or a salt thereof, phosphonic acid or a salt thereof, phosphoric acid or a salt thereof, carboxylic acid or a salt thereof, amino group of the formula —NR^{b}R^{c}, quaternary phosphonium salt of the formula —P⁺R^{b}R^{c}R^{d}X⁻, or quaternary ammonium salt of the formula —N⁺R^{b}R^{c}R^{d}X⁻, wherein each occurrence of R^{b}, R^{c}, and R^{d} is independently hydrogen, C₁-C₄ alkyl, hydroxy-substituted C₁-C₄ alkylene, C₁-C₄ alkoxy-substituted C₁-C₄ alkylene, amino(C₁-C₄ alkylene), or N,N-(di-C₁-C₃ alkyl)amino-C₁-C₄ alkylene.

### Embodiment 15

The method of Embodiment 13 or Embodiment 14, wherein each occurrence of R¹ is independently a hydroxyl, sulfonic acid or a salt thereof, phosphonic acid or a salt thereof, phosphoric acid or a salt thereof, carboxylic acid or a salt thereof, quaternary phosphonium salt of the formula —P⁺R^{b}R^{c}R^{d}X⁻, or quaternary ammonium salt of the formula —N⁺R^{b}R^{c}R^{d}X⁻, wherein each occurrence of R^{b}, R^{c}, and R^{d} is independently hydrogen or C₁-C₄ alkyl.

### Embodiment 16

The method of any of Embodiments 1 to 15, wherein the hydroxylamine-containing compound is reacted with the aromatic carboxy-aldehyde in an amount of 0.001 to 10 weight percent, based on the weight of the aromatic carboxy-aldehyde.

### Embodiment 17

The method of any of Embodiments 1 to 16, wherein the aromatic carboxy-aldehyde has the structure

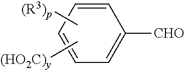

wherein p is 0 to 4, y is 1 to 5, provided that p+y=1, 2, 3, 4, or 5, and each occurrence of R³ is independently halogen, cyano, thiocyanato, nitro, C₁-C₅ alkyl, C₂-C₅ alkenyl, C₁-C₅ alkoxy, C₁-C₅ alkylthio, C₃-C₅ cycloalkyl, C₂-C₅ acyl, C₆-C₁₂ aryl, C₆-C₁₂ aryloxy, C₃-C₈ heteroaryl, or carbamoyl.

### Embodiment 18

The method of any of Embodiments 1 to 17, wherein the aromatic carboxy-aldehyde comprises 2-carboxybenzaldehyde, 3-carboxybenzaldehyde, 4-carboxybenzaldehyde, or a combination comprising at least one of the foregoing.

### Embodiment 19

The method of any of Embodiments 1 to 18, wherein the aromatic acid comprises 2, 3, 4, or 5 carboxylic acid groups.

### Embodiment 20

The method of any of Embodiments 1 to 19, wherein the aromatic acid or corresponding anhydride is phthalic acid, phthalic anhydride, terephthalic acid, isophthalic acid, or a combination comprising at least one of the foregoing.

### Embodiment 21

The method of any of Embodiments 1 to 20, wherein the nitrone has a water solubility at 25° C. of greater than 1 g/100 mL, preferably greater than 2 g/100 mL, preferably greater than 5 g/100 mL.

### Embodiment 22

The method of any of Embodiments 1 to 21, wherein the nitrone has the structure

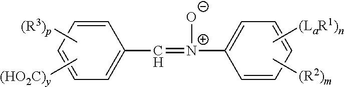

wherein p is 0 to 4, y is 1 to 5, provided that p+y=1, 2, 3, 4, or 5, n is 1 to 5 and m is 0 to 4, provided that n+m=1, 2, 3, 4, or 5, a is 0 or 1, L is a C₁-C₃ alkylene or phenylene, each occurrence of R¹ is independently a hydroxyl, sulfhydryl, sulfonyl, C₂-C₅ alkoxycarbonyl, C₂-C₅ alkylcarbonyloxy, sulfonic acid, carboxamide or a salt thereof, tosylate, mesylate, phosphonic acid or a salt thereof, phosphoric acid or a salt thereof, carboxylic acid or a salt thereof, C₂-C₄ alkylamido, C₁-C₄ alkylthio, ether of the formula —OR^{a}, amino group of the formula —NR^{b}R^{c}, quaternary phosphonium salt of the formula —P⁺R^{b}R^{c}R^{d}X—, or quaternary ammonium salt of the formula —N⁺R^{b}R^{c}R^{d}X⁻; wherein each occurrence of R^{a} is independently C₁-C₄ alkyl or —(CᵣH₂ᵣO)ₛCᵣH₂ᵣ₊₁ wherein each occurrence of r is independently 1 to 3 and each occurrence of s is independently 1 to 12, wherein each occurrence of R^{b}, R^{c}, and R^{d} is independently hydrogen, C₁-C₄ alkyl, C₂-C₄ alkenyl, hydroxy-substituted C₁-C₄ alkylene, C₁-C₄ alkoxy-substituted C₁-C₄ alkylene, amino(C₁-C₄ alkylene), N,N-(di-C₁-C₃ alkyl)amino-C₁-C₄ alkylene, and each occurrence of X is an organic or inorganic ion, each occurrence of R² is independently halogen, cyano, thiocyanato, nitro, C₁-C₃ alkyl, C₂-C₃ alkenyl, C₁-C₃ alkoxy, C₁-C₃ alkylthio, C₃-C₅ cycloalkyl, C₂-C₅ acyl, C₃-C₈ heteroaryl, or carbamoyl, and each occurrence of R³ is independently halogen, cyano, thiocyanato, nitro, C₁-C₅ alkyl, C₁-C₅ alkenyl, C₁-C₅ alkoxy, C₁-C₅ alkylthio, C₃-C₅ cycloalkyl, C₂-C₅ acyl, C₆-C₁₂ aryl, C₆-C₁₂ aryloxy, C₃-C₈ heteroaryl, or carbamoyl.

### Embodiment 23

An aromatic acid or the corresponding anhydride prepared according to the method of any of Embodiments 1 to 22.

### Embodiment 24

A method for purifying a phenyl dicarboxylic acid or the corresponding anhydride, comprising: oxidizing a xylene to provide a stream comprising the phenyl dicarboxylic acid and the corresponding carboxybenzaldehyde and toluic acid contaminants; reacting a hydroxylamine-containing compound with the carboxybenzaldehyde in the stream to form a reaction mixture comprising the corresponding nitrone, wherein the nitrone is water soluble; and crystallizing the phenyl dicarboxylic acid or the corresponding anhydride from water to provide the purified phenyl dicarboxylic acid or the corresponding anhydride.

### Embodiment 25

The method of Embodiment 24, wherein the phenyl dicarboxylic acid or the corresponding anhydride comprises: less than 0.5 wt. % of the carboxybenzaldehyde, preferably less than 0.25 wt. % of the carboxybenzaldehyde, more preferably less than 0.05 wt. % of carboxybenzaldehyde; and less than of less than 0.2 wt. % of the toluic acid, preferably less than 0.1 wt. % of the toluic acid, more preferably less than 0.05 wt. % of the toluic acid.

### Embodiment 26

A phenyl dicarboxylic acid prepared according to the method of Embodiment 24 or Embodiment 25.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention.

Unless otherwise specified herein, any reference to standards, testing methods and the like, such as ASTM D1003, ASTM D3359, ASTM D3363, refer to the standard, or method that is in force at the time of filing of the present application.

All ranges disclosed herein are inclusive of the endpoints, and the endpoints are independently combinable with each other. “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term. Reference throughout the specification to “one embodiment”, “another embodiment”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “+10%” means that the indicated measurement can be from an amount that is minus 10% to an amount that is plus 10% of the stated value.

As used herein, the term “hydrocarbyl” and “hydrocarbon” refers broadly to a substituent comprising carbon and hydrogen, optionally with 1 to 3 heteroatoms, for example, oxygen, nitrogen, halogen, silicon, sulfur, or a combination thereof; “alkyl” refers to a straight or branched chain, saturated monovalent hydrocarbon group; “alkylene” refers to a straight or branched chain, saturated, divalent hydrocarbon group; “alkylidene” refers to a straight or branched chain, saturated divalent hydrocarbon group, with both valences on a single common carbon atom; “alkenyl” refers to a straight or branched chain monovalent hydrocarbon group having at least two carbons joined by a carbon-carbon double bond; “cycloalkyl” refers to a non-aromatic monovalent monocyclic or multicylic hydrocarbon group having at least three carbon atoms, “cycloalkenyl” refers to a non-aromatic cyclic divalent hydrocarbon group having at least three carbon atoms, with at least one degree of unsaturation; “aryl” refers to an aromatic monovalent group containing only carbon in the aromatic ring or rings; “arylene” refers to an aromatic divalent group containing only carbon in the aromatic ring or rings; “alkylaryl” refers to an aryl group that has been substituted with an alkyl group as defined above, with 4-methylphenyl being an exemplary alkylaryl group; “arylalkyl” refers to an alkyl group that has been substituted with an aryl group as defined above, with benzyl being an exemplary arylalkyl group; “acyl” refers to an alkyl group as defined above with the indicated number of carbon atoms attached through a carbonyl carbon bridge (—C(═O)—); “alkoxy” refers to an alkyl group as defined above with the indicated number of carbon atoms attached through an oxygen bridge (—O—); and “aryloxy” refers to an aryl group as defined above with the indicated number of carbon atoms attached through an oxygen bridge (—O—).

Unless otherwise indicated, each of the foregoing groups can be unsubstituted or substituted, provided that the substitution does not significantly adversely affect synthesis, stability, or use of the compound. The term “substituted” as used herein means that at least one hydrogen on the designated atom or group is replaced with another group, provided that the designated atom's normal valence is not exceeded. When the substituent is oxo (i.e., ═O), then two hydrogens on the atom are replaced. Combinations of substituents and/or variables are permissible provided that the substitutions do not significantly adversely affect synthesis or use of the compound. Exemplary groups that can be present on a “substituted” position include, but are not limited to, cyano; hydroxyl; nitro; azido; alkanoyl (such as a C₂₋₆ alkanoyl group such as acyl); carboxamido; C₁₋₆ or C₁₋₃ alkyl, cycloalkyl, alkenyl, and alkynyl (including groups having at least one unsaturated linkages and from 2 to 8, or 2 to 6 carbon atoms); C₁₋₆ or C₁₋₃ alkoxyl; C₆₋₁₀ aryloxy such as phenoxy; C₁₋₆ alkylthio; C₁₋₆ or C₁₋₃ alkylsulfinyl; C₁₋₆ or C₁₋₃ alkylsulfonyl; aminodi(C₁₋₆ or C₁₋₃)alkyl; C₆₋₁₂ aryl having at least one aromatic rings (e.g., phenyl, biphenyl, naphthyl, or the like, each ring either substituted or unsubstituted aromatic); C₇₋₁₉ arylalkyl having 1 to 3 separate or fused rings and from 6 to 18 ring carbon atoms; or arylalkoxy having 1 to 3 separate or fused rings and from 6 to 18 ring carbon atoms, with benzyloxy being an exemplary arylalkoxy.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

I/We claim:

1. A method of purifying a stream comprising an aromatic carboxy-aldehyde and an aromatic acid or the corresponding anhydride, comprising:
reacting a hydroxylamine-containing compound with the aromatic carboxy-aldehyde in the stream to form a reaction mixture comprising the corresponding nitrone, wherein the nitrone is water soluble;
adding an aqueous solvent to the reaction mixture, in an amount and under conditions effective to solubilize the nitrone but not the aromatic acid or the corresponding anhydride; and
separating the solubilized nitrone from the non-solubilized aromatic acid.

2. The method of claim 1, wherein the adding the solvent and the separating comprises crystallizing the aromatic acid or the corresponding anhydride from the aqueous solvent.

3. The method of claim 2, wherein the crystallizing further removes the corresponding toluic acid from the aromatic acid or the corresponding anhydride.

4. The method of claim 2, wherein the crystallized aromatic acid or corresponding anhydride has less than 2 wt. % of the corresponding toluic acid.

5. The method of claim 1, wherein the separating is by filtering, decanting, or centrifuging the reaction mixture to separate the aromatic acid or the corresponding anhydride from the solvent comprising the solubilized nitrone.

6. The method of claim 1, wherein the separated aromatic acid or corresponding anhydride has a purity of 90-99%.

7. The method of claim 2, wherein after the reacting to form the nitrone or the separating, the aromatic acid or corresponding anhydride has a yellowness index of less than or equal to 10 as determined according to ASTM D1209.

8. The method of claim 2, wherein after the reacting to form the nitrone or the separating, the aromatic acid or corresponding anhydride has a delta-Y value of at least 5% lower than the aromatic acid or corresponding anhydride before the reacting, as determined according to ASTM D1209.

9. The method of claim 1, wherein the hydroxylamine-containing compound has the structure 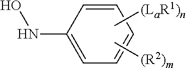
wherein
n is 1 to 5 and m is 0 to 4, provided that n+m=1, 2, 3, 4, or 5,
a is 0 or 1,
L is a C₁-C₃ alkylene or phenylene,
each occurrence of R¹ is independently a hydroxyl, sulfhydryl, sulfonyl, sulfonic acid or a salt thereof, tosylate, mesylate, C₂-C₅ alkoxycarbonyl, C₂-C₅ alkylcarbonyloxy, C₂-C₅ carboxamide, alkoxycarbonyl, C₂-C₅ alkylcarbonyloxy, phosphonic acid or a salt thereof, phosphoric acid or a salt thereof, carboxylic acid or a salt thereof, C₁-C₄ alkylthio, ether of the formula —OR^{a}, amino of the formula —NR^{b}R^{c}, aminocarboxy of the formula —C(O)NR^{b}R^{c}, quaternary phosphonium salt of the formula —P⁺R^{b}R^{c}R^{d}X⁻, or quaternary ammonium salt of the formula —N⁺R^{b}R^{c}R^{d}X⁻,
wherein each occurrence of R^{a} is independently C₁-C₄ alkyl or —(CᵣH₂ᵣO)ₛCᵣH₂ᵣ₊₁ wherein each occurrence of r is independently 1 to 3 and each occurrence of s is independently 1 to 12,
wherein each occurrence of R^{b}, R^{c}, and R^{d} is independently hydrogen, C₁-C₄ alkyl, hydroxy-substituted C₁-C₄ alkylene, C₁-C₄ alkoxy-substituted C₁-C₄ alkylene, amino(C₁-C₄ alkylene), N,N-(di-C₁-C₃ alkyl)amino-C₁-C₄ alkylene, and
each occurrence of X is an organic or inorganic ion, and
each occurrence of R² is independently halogen, cyano, thiocyanato, nitro, C₁-C₃ alkyl, C₂-C₃ alkenyl, C₁-C₃ alkoxy, C₁-C₃ alkylthio, C₃-C₅ cycloalkyl, C₂-C₅ acyl, C₃-C₈ heteroaryl, or carbamoyl.

10. The method of claim 9, wherein each occurrence of R¹ is independently a hydroxyl, sulfonic acid or a salt thereof, phosphonic acid or a salt thereof, phosphoric acid or a salt thereof, carboxylic acid or a salt thereof, amino group of the formula —NR^{b}R^{c}, quaternary phosphonium salt of the formula —P⁺R^{b}R^{c}R^{d}X⁻, or quaternary ammonium salt of the formula —N⁺R^{b}R^{c}R^{d}X⁻, wherein each occurrence of R^{b}, R^{c}, and R^{d} is independently hydrogen, C₁-C₄ alkyl, hydroxy-substituted C₁-C₄ alkylene, C₁-C₄ alkoxy-substituted C₁-C₄ alkylene, amino(C₁-C₄ alkylene), or N,N-(di-C₁-C₃ alkyl)amino-C₁-C₄ alkylene.

11. The method of claim 1, wherein the hydroxylamine-containing compound is reacted with the aromatic carboxy-aldehyde in an amount of 0.001 to 10 weight percent, based on the weight of the aromatic carboxy-aldehyde.

12. The method of claim 1, wherein the aromatic carboxy-aldehyde has the structure 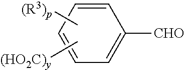
wherein
p is 0 to 4, y is 1 to 5, provided that p+y=1, 2, 3, 4, or 5, and
each occurrence of R³ is independently halogen, cyano, thiocyanato, nitro, C₁-C₅ alkyl, C₂-C₅ alkenyl, C₁-C₅ alkoxy, C₁-C₅ alkylthio, C₃-C₅ cycloalkyl, C₂-C₅ acyl, C₆-C₁₂ aryl, C₆-C₁₂ aryloxy, C₃-C₈ heteroaryl, or carbamoyl.

13. The method of claim 1, wherein the aromatic carboxy-aldehyde comprises 2-carboxybenzaldehyde, 3-carboxybenzaldehyde, 4-carboxybenzaldehyde, or a combination comprising at least one of the foregoing.

14. The method of claim 1, wherein the aromatic acid or corresponding anhydride is phthalic acid, phthalic anhydride, terephthalic acid, isophthalic acid, or a combination comprising at least one of the foregoing.

15. The method of claim 1, wherein the nitrone has a water solubility at 25° C. of greater than 1 g/100 mL.

16. The method of claim 1, wherein the nitrone has the structure 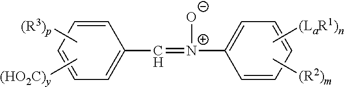
wherein
p is 0 to 4, y is 1 to 5, provided that p+y=1, 2, 3, 4, or 5,
n is 1 to 5 and m is 0 to 4, provided that n+m=1, 2, 3, 4, or 5,
a is 0 or 1,
L is a C₁-C₃ alkylene or phenylene,
each occurrence of R¹ is independently a hydroxyl, sulfhydryl, sulfonyl, C₂-C₅ alkoxycarbonyl, C₂-C₅ alkylcarbonyloxy, sulfonic acid, carboxamide or a salt thereof, tosylate, mesylate, phosphonic acid or a salt thereof, phosphoric acid or a salt thereof, carboxylic acid or a salt thereof, C₂-C₄ alkylamido, C₁-C₄ alkylthio, ether of the formula —OR^{a}, amino group of the formula —NR^{b}R^{c}, quaternary phosphonium salt of the formula —P⁺R^{b}R^{c}R^{d}X⁻, or quaternary ammonium salt of the formula —N⁺R^{b}R^{c}R^{d}X⁻,
wherein each occurrence of R^{a} is independently C₁-C₄ alkyl or —(CᵣH₂ᵣO)ₛCᵣH₂ᵣ₊₁ wherein each occurrence of r is independently 1 to 3 and each occurrence of s is independently 1 to 12,
wherein each occurrence of R^{b}, R^{c}, and R^{d} is independently hydrogen, C₁-C₄ alkyl, C₂-C₄ alkenyl, hydroxy-substituted C₁-C₄ alkylene, C₁-C₄ alkoxy-substituted C₁-C₄ alkylene, amino(C₁-C₄ alkylene), N,N-(di-C₁-C₃ alkyl)amino-C₁-C₄ alkylene, and
each occurrence of X is an organic or inorganic ion,
each occurrence of R² is independently halogen, cyano, thiocyanato, nitro, C₁-C₃ alkyl, C₂-C₃ alkenyl, C₁-C₃ alkoxy, C₁-C₃ alkylthio, C₃-C₅ cycloalkyl, C₂-C₅ acyl, C₃-C₈ heteroaryl, or carbamoyl, and
each occurrence of R³ is independently halogen, cyano, thiocyanato, nitro, C₁-C₅ alkyl, C₁-C₅ alkenyl, C₁-C₅ alkoxy, C₁-C₅ alkylthio, C₃-C₅ cycloalkyl, C₂-C₅ acyl, C₆-C₁₂ aryl, C₆-C₁₂ aryloxy, C₃-C₈ heteroaryl, or carbamoyl.

17. An aromatic acid or the corresponding anhydride prepared according to the method claim 1.

18. A method for purifying a phenyl dicarboxylic acid or the corresponding anhydride, comprising:
oxidizing a xylene to provide a stream comprising the phenyl dicarboxylic acid and the corresponding carboxybenzaldehyde and toluic acid contaminants;
reacting a hydroxylamine-containing compound with the carboxybenzaldehyde in the stream to form a reaction mixture comprising the corresponding nitrone, wherein the nitrone is water soluble; and
crystallizing the phenyl dicarboxylic acid or the corresponding anhydride from water to provide the purified phenyl dicarboxylic acid or the corresponding anhydride.

19. The method of claim 18, wherein the phenyl dicarboxylic acid or the corresponding anhydride comprises:
less than 0.5 wt. % of the carboxybenzaldehyde; and
less than of less than 0.2 wt. % of the toluic acid.

20. A phenyl dicarboxylic acid prepared according to the method of claim 18.
